(19) United States
(12) Patent Application Publication
(10) Pub. No.: US 2021/0285939 A1
(43) Pub. Date: Sep. 16, 2021

(54) ELECTROPOLYMERIZED ALLERGEN DETECTION DEVICE AND METHODS OF USE THEREOF

(71) Applicant: Allergy Amulet, Inc., Madison, WI (US)

(72) Inventors: Srikanth Rao Agnihotra, Tyngsboro, MA (US); Madanodaya Sundhoro, Lowell, MA (US); Brent Amberger, Dracut, MA (US); Abigail Barnes, Madison, WI (US); Joseph BelBruno, Hanover, NH (US); Nazir Khan, Boston, MA (US); Keenan Augustus, Nashua, NH (US)

(73) Assignee: Allergy Amulet, Inc., Madison, WI (US)

(21) Appl. No.: 17/290,737

(22) PCT Filed: Oct. 30, 2019
(86) PCT No.: PCT/US2019/058833

§ 371 Date: Apr. 30, 2021

Related U.S. Application Data
(60) Provisional application No. 62/754,389, filed on Nov. 1, 2018.

(51) Int. Cl. G01N 33/53; G01N 33/02; G01N 33/04; G01N 33/14; C08G 73/06; G01N 27/327; G01N 27/48

(52) U.S. Cl. CPC G01N 33/5308; G01N 33/02; G01N 33/04; G01N 2600/00; C08G 73/0611; G01N 27/3277; G01N 27/48; G01N 33/14

(57) ABSTRACT

An allergen detection device that includes a sensor comprising a circuit board, an electropolymerized molecularly imprinted polymer film (MIP) that includes receptor sites imprinted in a first surface of the polymer, the receptor sites configured to accept a trace molecule of an allergen, and an electropolymerized non-imprinted polymer film. The sensor is configured to detect the presence of the trace molecule upon binding to one or more of the receptor sites on the MIP.

### PRIORITY

This application claims the benefit of priority to U.S. Provisional Application No. 62/754,389, filed on Nov. 1, 2018, and which is incorporated herein by reference in its entirety for all purposes.

### BACKGROUND

Many people suffer from allergies or are intolerant to various ingredients/compounds in consumable products such as foods, drinks, and cosmetics of various types. While the severity of the reactions varies, many reactions can cause severe gastrointestinal distress and can even be fatal. Preventing the inadvertent exposure to such ingredients/compounds is a concern for many. Present allergen-detection tools for assisting individuals with avoiding exposure generally require sophisticated technology and expertise. These tools are also typically too bulky for individuals to use at the point of consumption of the consumable product.

### SUMMARY

In one aspect, an allergen detection device is provided. The device may include a sensor that includes an electropolymerized molecularly imprinted polymer (MIP) film comprising receptor sites imprinted in a first surface of the polymer, wherein the receptor sites are configured to accept a trace molecule of an allergen; and an electropolymerized non-imprinted polymer (NIP) film (i.e., control). In any embodiment, the sensor may be configured to detect the presence of the trace molecule upon binding to one or more of the receptor sites on the MIP. In any embodiment, the trace molecule may be in a consumable good. In any embodiment, the device may further include a first electrochemical chip, wherein the first electrochemical chip comprises the MIP film and/or a second electrochemical chip, wherein the second electrochemical chip comprises the NIP film. In any embodiment, the device may further include a circuit board (e.g., printed circuit board) comprising the first electrochemical chip and the second electrochemical chip.

In any embodiment, the device may further include a processing device. Commonly, the processing device may be configured to communicatively couple to the sensor and may be configured to determine an electric current difference between the MIP film and the NIP film. In any of the above embodiments, the processing device may determine the presence of the allergen when the electric current of the MIP film is greater than the electric current of the NIP film. In any of the above embodiments, the processing device may determine the presence of the allergen when the electric current of the MIP film is lower than the electric current of the NIP film.

In any of the above embodiments, the device may also include a body, wherein the body comprises a capsule that encapsulates a solvent; and a chamber for mixing the solvent with the consumable good. In any embodiment, the device may further include a substrate with a consumable good sample on the surface configured for insertion into the chamber. In any of the above embodiments, the device may further include a locking mechanism for locking the substrate into the chamber. In any of the above embodiments, the locking mechanism may include a ramp adjacent to the chamber. In any of the above embodiments, the ramp is configured to puncture the capsule releasing the solvent into the chamber. In any of the above embodiments, the device further comprises a recess configured to house the sensor.

### BRIEF DESCRIPTION OF THE DRAWINGS

FIG. 1 shows a printed circuit board 1, according to one embodiment.

FIG. 2 shows a printed circuit board 2, according to one embodiment.

FIG. 3 shows a printed circuit board 3, according to one embodiment.

FIG. 4 shows a processing device 1, according to one embodiment. Green, red, and yellow lights are provided to reveal the results of the allergen test. Green light shows the absence of the allergen. Red light shows the presence of the allergen. Flashing yellow light shows that reading is in progress, and a stable yellow light represent an inconclusive reading.

FIG. 5 shows a wearable processing device 1, according to one embodiment.

FIG. 6 shows a processing device 2, according to one embodiment. Green, red, and yellow lights are provided to reveal the results of the allergen test. Green light shows the absence of the allergen. Red light shows the presence of the allergen. Flashing yellow light shows that reading is in progress, and a stable yellow light represent an inconclusive reading.

FIG. 7 shows a wearable processing device 2, according to one embodiment.

FIG. 8 shows a processing device 3, according to one embodiment.

FIG. 9 shows a wearable processing device 3, according to one embodiment.

FIG. 10 shows a processing device 4, according to one embodiment. Green, red, and yellow lights are provided to reveal the results of the allergen test. Green light shows the absence of the allergen. Red light shows the presence of the allergen. Flashing yellow light shows that reading is in progress, and a stable yellow light represent an inconclusive reading.

FIG. 11 shows a wearable processing device 4, according to one embodiment.

FIG. 12 shows the outside view of a device for allergen detection, according to one embodiment.

FIG. 13 shows the inside view of a device for allergen detection, according to one embodiment.

FIG. 14 shows the overhead view of a device with a ramp, according to one embodiment.

FIG. 15 shows the overhead view of a device with a ramp and depicts how inserting a substrate (e.g., test strip) releases a solvent into the chamber, according to one embodiment.

FIG. 16 shows overhead and side view of the device with a detector for allergen detection, according to one embodiment.

### DETAILED DESCRIPTION

Daily interactions with consumable goods can be challenging for individuals with an allergy or intolerance to ingredients commonly used is such materials. For example, eating foods prepared by others. The present technology provides a fast and portable allergen and/or ingredient detection device enabling users to directly sample the consumable good for unwanted ingredients. The device provides individuals the ability to feel safer about the products they use and the foods they eat. The allergen(s) and/or ingredients may be detected by inserting a substrate (e.g., a single-use test strip) into a liquid or solid consumable good sample. The substrate may then be inserted into the chamber of the device, shaken, and connected to a processing device. The device includes a sensor comprising an MIP. MIPs are polymer compositions having synthetic cavities, or binding pockets, designed to bind to trace molecules. If the trace molecule is present in the tested sample, binding occurs, i.e. the target allergen or a molecule indicative of the target allergen/ingredient fills the binding pocket in the MIP, and the processing device then detects a measurable interaction, alerting the user to the presence of the trace molecule within a short period of time (e.g., seconds). If no binding occurs, the processing device signals that the trace molecule was not detected.

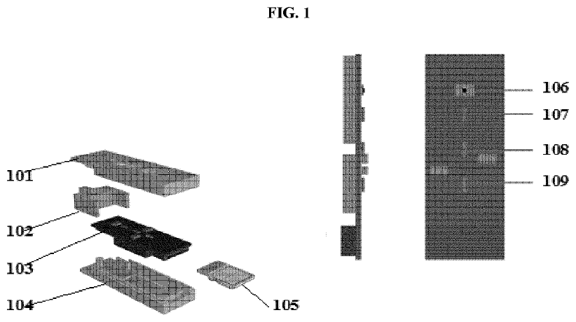

The processing device can be configured as a wearable device, or it may be integrated into everyday products that users can keep on their person. With an accompanying software application (i.e. “app”), users can track and upload tests, connect with other allergic individuals, and store and share important information including, but not limited to, emergency contacts. FIGS. 1-11 show illustrative embodiments of the processing device.

The following terms are used throughout this disclosure, as defined below.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Unless otherwise indicated, numeric ranges, for instance as in “from 2 to 10,” are inclusive of the numbers defining the range (e.g., 2 and 10).

Unless otherwise indicated, ratios, percentages, parts, and the like are by weight.

As used herein a “trace molecule” refers to molecules that are suitable for detecting the presence of an allergen but may not necessarily be allergens themselves. For example, the trace molecule may be the allergen itself, epitope of an allergen, molecule that is commonly present with an allergen, a subunit of an allergen, a derivative of an allergen, or a combination of two or more thereof.

As used herein “allergen” refers to both allergy and intolerant inducing substances. A true allergy causes an immune system reaction that affects numerous organs in the body and can cause a range of symptoms. In some cases, an allergic reaction can be severe or life-threatening. In comparison, intolerance symptoms are generally less serious and often limited to digestive problems. Nonlimiting examples of intolerances include absence of an enzyme needed to fully digest a consumable (e.g., food or drink), irritable bowel syndrome, sensitivity to an additive, recurring stress or psychological factors, and Celiac disease. An example of an absence of an enzyme is lactose intolerance. Irritable bowel syndrome is a chronic condition that may cause cramping, constipation, and/or diarrhea. An example of sensitivity to an additive are sulfites commonly used to preserve food and drinks. Celiac disease has some features of a true food allergy because it involves the immune system, however, symptoms are mostly gastrointestinal, and people with celiac disease are not at risk of anaphylaxis.

As used herein “consumable goods” refers to goods that are intended to be consumed. In any embodiment, the consumable good may include food, drink, cosmetic (i.e., skincare or haircare product such as cleanser, moisturizer, shampoo, conditioner, makeup, and/or perfume), or a combination of two or more thereof. A consumable good may include one or more trace molecules. A trace molecule may be present in any of a variety of items that may be a target for detecting an allergen. For example, a trace molecule may be present in the consumable good itself or in an item the consumable good has come into contact. For example, an item that a food has come into contact with (e.g., a serving utensil, a table, etc.) or an item that a skincare product has come into contact (e.g., plastic packaging). Consumable goods that may include a trace molecule may come in a variety of forms including, but not limited to, a solid, a liquid, a gas, a suspension, an emulsification, and any combinations thereof. Example solid consumable goods include, but are not limited to, a solid food (e.g., a bread, a nut), a plate, a table, a utensil, solid makeup (e.g., eyeshadow or lipstick), and any combinations thereof. Example liquid consumable goods include, but are not limited to, a liquid food, a beverage (e.g., a soda, milk, a juice), a food extract, shampoo, perfume, and any combinations thereof. Examples of suspension consumable goods include, but are not limited to, a consumable good suspended in air (e.g., a composition in particulate form), a consumable good suspended in a solvent (e.g., sprayable hair product), and any combinations thereof. Examples of emulsion consumable goods include, but are not limited to, moisturizer emulsions (e.g., lotion), conditioner emulsions, cleanser emulsions, and any combinations thereof.

As used herein “electric current” or “current” refers to the flow of electric charge. In any embodiment, electric current may be directly measured, determined through a mathematical construct (e.g., average or weighted average), or a combination thereof. Commonly, current measurements may be taken by any known electrochemical experiment including, but not limited to, cyclic voltammetry (CV), linear sweep voltammetry, square wave voltammetry, differential pulse voltammetry, amperometry, or a combination thereof. In any embodiment, electric current may be measured at the maximum, minimum, or average current between pre-set voltage values and/or between inflection points. These measurements may be taken before and after incubation, continuously, or with some degree of mid-incubation data points. In any embodiment, changes between the pre-incubation and post-incubation measurements for the MIP and NIP control films indicate the presence or absence of the target analyte. Pre-incubation measurements may be stored in memory as a set value or as bar codes, QR codes, or in flash drives. If measurements are taken in a combined sample/electrolyte incubation solution, continuous/intermediate scans may be taken as well. Many uses can be envisioned for intermediate data points, including checking the veracity of the pre-incubation and post-incubation measurements to enhance test confidence. Other data, such as points of maximum current, average current, an inflection point, and/or at a pre-defined voltage during either the oxidative or reductive phase of a CV may be used to compare pre- and post-incubation results. This data may be used in place of, or to supplement, peak current measurements. In any embodiment, electric currents may be derived from a single cycle of an electrochemical experiment or an average or weighted average from two or more cycles.

Allergens may include, but are not limited to, animal products, grains (e.g., gluten), vegetables, fruits, dairy products, fish, beverages, legumes, chocolates, synthetic food chemicals (e.g., monosodium glutamate (MSG), and any combinations of two or more thereof. In one example, an allergen may include a food protein. Due to the importance of peanut allergies, a peanut-related allergen is used in an exemplary fashion in this disclosure. It is contemplated that other allergens may replace the discussed peanut-related allergen in the example, embodiment, implementation or other aspect of the disclosure. One way to test for the presence of a peanut-related allergen is to test for a peanut protein allergen. Examples of a peanut protein include, but are not limited to, Arachis hypogaea allergen 1 (Ara h1), Arachis hypogaea allergen 2 (Ara h2), Arachis hypogaea allergen 3 (Ara h3), Arachis hypogaea allergen 4 (Ara h4), Arachis hypogaea allergen 5 (Ara h5), Arachis hypogaea allergen 6 (Ara h6), Arachis hypogaea allergen 7 (Ara h7), Arachis hypogaea allergen 8 (Ara h8), Arachis hypogaea allergen 9 (Ara h9), Arachis hypogaea allergen 10 (Ara h10), Arachis hypogaea allergen 11 (Ara h11), Arachis hypogaea allergen 12 (Ara h12), Arachis hypogaea allergen 13 (Ara h13), Arachis hypogaea allergen 14 (Ara h14), Arachis hypogaea allergen 15 (Ara h15), Arachis hypogaea allergen 16 (Ara h16), Arachis hypogaea allergen 17 (Ara h17), peanut agglutinin (PNA), and combinations of any two or more thereof. Further specific examples of allergens are listed in Table 1 below.

TABLE 1
List of allergens:

Ara H1 epitopes:
NNPFYFPSR
SFNLDEGHALR
NTLEAAFNAEFNEIR
VLLEENAGGEQEER
DLAFPGSGEQVEK
GTGNLELVAVR
Ara H2 epitopes:
CMCEALQQIMENQSDR
RQQWELQGDR
Ara H3/H4 epitopes:
SPDIYNPQAGSLK
SQSENFEYVAFK
RPFYSNAPQEIFIQQGR
WLGLSAEYGNLYR
Milk
Allergenic proteins Bos d 2, 3, 4, 5, 6, 7, 8, 9, 10, 11, 12
Soy
Allergenic proteins (Gly m 1, 2, 3, 4, 5, 6, 7, 8)
Wheat
Allergenic proteins (Tri a 12, 14, 15, 17, 18, 19, 20, 21, 25, 26, 27, 28, 29, 30,
31, 32, 33, 34, 35, 36, 37, 39, 40, 41, 42, 43, 44, 45)
Fish
Allergenic proteins: Lep w 1, Lep s 1, Pon 14, Pon 17, Pro c 1, Pro c 2, Pro c 5,
Pro c 8, Seb m 1, Xip g 1, Onc k 5, Sal s 1, 2, 3, Thu a 1, 2, 3, Ras k 1, Clu h 1,
Cyp c 1, Gad c 1, Gad m 1, 2, 3, Lat c 1, Onc ml, Ore m 4, Sal s 1, 2, 3, Sar sa 1,
Seb m 1
Shellfish
Allergenic proteins: Cra q 1 (Pacific Oyster), Hal I 1 (Jade tiger abalone), Hal
m 1 (abalone), Hel as 1 (brown garden snail), Sac q 1 (Sydney rock oyster),
Tod p 1 (Japanese flying squid), Art fr 5 (Brine shrimp), Cra c 1, 2, 4, 5, 6, 8
(North sea shrimp), Lit v 1, 2, 3, 4 (white shrimp), Met e 1 (shrimp), Pan b 1
(Northern shrimp), Pen a 1 (brown shrimp), Pen i 1 (shrimp), Pen m 1, 2, 3, 4,
6 (black tiger shrimp), Cha f 1 (crab), Por p 1 (blue swimmer crab), Scy p 2, 4,
8 (mud crab), Vesp c 1, 5 ( European hornet), Hom a 1, 3, 6 (American
lobster), Pan s 1 (spiny lobster), Mac r 1 (giant fresh water prawn), Mel 1 1
(king prawn), Pon 14, 7 (narrow-clawed crayfish), Pro c 1, 2, 5, 8 (red swamp
crayfish)
Egg
Allergenic proteins: Gal d 1, 2, 3, 4, 5, 6, 7, 8, 9, 10
Nuts
Allergenic proteins: Ber e 1, 2 (brazil nut), Cas s 1, 5, 8, 9 (chestnut), Cor a 1, Ana
o 1, 2, 3 (cashew), Pis v 1, 2, 3, 4, 5 (pistachio), Pm du 3, 4, 5, 6 (almond)
Almond
Almond paste
Anacardium nuts
Anacardium occidentale (Anacardiaceae)[botanical name, Cashew]
Artificial nuts
Beech nut
Brazil nut
Bertholletia excelsa (Lecythidaceae)[botanical name, Brazil nut]
Bush nut
Butternut
Butyrospermum Parkii [botanical name, Shea nut]
Canarium ovatum Engl. in A. DC. (Burseraceae)[botanical name, Pili nut]
Caponata
Carya illinoensis (Juglandaceae)[botanical name, Pecan]
Carya spp. (Juglandaceae)[botanical name, Hickory nut]
Cashew
Castanea pumila (Fagaceae)[botanical name, Chinquapin]
Castanea spp. (Fagaceae)[botanical name, Chestnut (Chinese, American,
European, Seguin)]
Chestnut (Chinese, American, European, Seguin)
Chinquapin
Cocos nucifera L. (Arecaceae (alt. Palmae))[botanical name, Coconut]
Corylus spp. (Betulaceae)[botanical name, Filbert/hazelnut]
Filbert
Fagus spp. (Fagaceae)[botanical name, beech nut]
Gianduja
Ginko nut
Ginkgo biloba L. (Ginkgoaceae)[botanical name, Ginko nut]
Hazelnut
Heartnut
Hickory nut
Indian nut
Juglans cinerea (Juglandaceae)[botanical name, Butternut]
Juglans spp. (Juglandaceae)[botanical name, Walnut, Butternut, Heartnut]
Karite (shea nut)
Lichee nut
Litchi chinensis Sonn. Sapindaceae [botanical name, Lichee nut]
Lychee nut
Macadamia nut
Macadamia spp. (Proteaceae)[botanical name, Macadamia nut/Bush nut]
Mandelonas
Marzipan
Mashuga nuts
Nangai nuts
Natural nut extract (for example, almond extract)
Nougat
Nu-Nuts ®
Nut butters (e.g., Almond butter, Hazelnut butter, Brazil nut butter, Macadamia
nut butter, Pistachio nut butter, Shea nut butter, Karike butter, as well as other nut
butters)
Nut meal
Nutella ®
Nutmeat
Nut oil (e.g., Walnut oil as well as other nut oils)
Nut paste
Nut pieces
Pecan
Pigñolia
Pili nut
Pine nut
Pine nut (Indian, piñon, pinyon, pigndi, pigñolia, pignon nuts)
Pinon nut
Piñon or Piñon nut
Pinus spp. (Pineaceae)[botanical name, Pine nut/piiion nut]
Pistachio
Pistacia vera L. (Anacardiaceae)[botanical name, Pistachio]
Pralines
Prunus dulcis (Rosaceae)[bontanical name, almond]
Shea nut
Sheanut
Vitellaria paradoxa C.F. Gaertn. (Sapotaceae)[botanical name, Shea nut]
Walnut (English, Persian, Black, Japanese, California)

To enable convenient detection of the above listed allergens, the technology provided herein is an allergen detection device that may be wearable. The device may include a sensor that includes an electropolymerized MIP film comprising receptor sites imprinted in a first surface of the polymer, the receptor sites configured to accept/bind a trace molecule of an allergen; and an electropolymerized NIP film. In any embodiment, the sensor may be configured to detect the presence of the trace molecule upon binding to one or more of the receptor sites on the MIP film.

In any embodiment, the trace molecule may be a peanut allergen, tree nut allergen, milk allergen, egg allergen, wheat allergen, soy allergen, meat allergen, fish allergen, shellfish allergen, coconut allergen, or a combination of two or more thereof. In any embodiment, the trace molecule may be a nut allergen listed in Table 1. In any embodiment, the trace molecule may be a tree nut allergen (e.g., almond, almond paste, or a combination thereof). In any embodiment, the trace molecule may be a soy allergen. In any embodiment, the trace molecule may include a flavonoid, amygdalin, or a combination thereof. In any embodiment, the flavonoid may include an isoflavonoid, neoflavonoid, or derivatives thereof. In any embodiment, the isoflavonoid or derivative thereof may include isoflavones, isoflavonones, isoflavans, pterocarpans, rotenoids, or combinations of two or more thereof. In any embodiment, the trace molecule may include amygdalin, apigenin-6-arabinoside-8-glucoside, apigenin-6-glucoside-8-arabino side, arachin, biochanin A, catechin gallate, crysoeriol, cyanocobalamin, daidzein, daidzin,5-5′-dehydrodiferulic acid, 5-8′-dehydrodiferulic acid, 5,7-dihydroxychromone, 5,7, dimethoxyisoflavone, ferulic acid, galactose, genistein, genistin, 3-hydroxybiochanin A, isochlorogenic acid, isoferulic acid, juglone, lactose, lariciresinol, medioresinol, procyanidin B2, procyanidin C1, resveratrol, resveratrol 3-glucoside, secoisolariciresinol, syringaresinol, syringic acid, trans-sinapic acid.

In any embodiment, the MIP film may include receptor sites for the trace molecule. In any embodiment, the trace molecule may include an organic molecule. In any embodiment, the organic molecule may have a molecular weight less than about 5000 g/mol (including less than about 900 Daltons and less than about 500 Daltons). In any embodiment, the organic molecule may be selected from lactose, galactose, amygdalin, juglone, biochanin A, resveratrol daidzein, daidzin, genistein, genistin, or a combination of two or more thereof.

In any embodiment, the organic molecule may include a polypeptide, protein, epitope, aptamer, or a combination of two or more thereof. In any embodiment, the organic molecule may include at least one protein. In any embodiment, the organic molecule may include at least two different proteins. In any embodiment, the organic molecule may include at least one epitope. In any embodiment, the organic molecule may include at least two different epitopes. In any embodiment, the organic molecule may include at least one protein and at least one epitope.

In any embodiment, the organic molecule may not include cortisol, an amino acid, theophylline, and/or chlorpyrifos,

In any embodiment, the polymer of the MIP and/or NIP may include one or more polymerized monomers. In any embodiment, the monomers may include 3-aminophenyl boronic acid, 4-aminophenyl boronic acid, 2-hydroxyphenyl boronic acid, 3-hydroxyphenyl boronic acid, 4-hydroxyphenol boronic acid, pyrrole, polyaniline, thiophene, 3,4-ethylenedioxythiophene, phenylene diamine, phenyl boronic acid, p-aminothiophenol, aminophenol, p-phenyl phenylenediamine, o-toluidine, or combinations of any two or more thereof. The polymer may include a co-polymer of any two or more monomers and/or a polymer blend of any two or more polymers. In any embodiment, the polymer may include polymerized pyrrole. In any embodiment, the polymer of the MIP and NIP include the same polymerized monomers.

In any embodiment, the device may further include a first electrochemical chip, wherein the first electrochemical chip comprises the MIP film and/or a second electrochemical chip, wherein the second electrochemical chip comprises the NIP film. In any embodiment, the device may further include a circuit board (e.g., printed circuit board) comprising the first electrochemical chip and the second electrochemical chip. In any embodiment, the circuit board may include the first electrochemical chip and the second electrochemical chip. In any embodiment, the electrochemical chips may be sourced in any known manner including screen printing, inkjet printing, vapor deposition, lithography, or subtractive methods. In any embodiment, the circuit board may be width and thickness to fit a standard interface (e.g., SD, MicroSD, USB, or USB-C). In any embodiment, the circuit board that includes the electrochemical chips may further include a substrate. The substrate may include copper on a PCB material in an interdigitated pattern. In some embodiments, the copper may be laminated on one or both sides of the PCB material. Non-limiting examples of PCB material include FR4, bakelite, glass, plastic, rubber, cellulose, and the like. In any embodiment, the circuit board may be about 1 cm² in area and have an interdigit spacing of 300 μm. FIGS. 1-3 show illustrative example of circuit boards.

In any embodiment, the first electrochemical chip may include a working electrode, a counter electrode, and a reference electrode. In any embodiment, the second electrochemical chip may include a working electrode, a counter electrode, and reference electrode. In any embodiment, the working, counter, and/or reference electrode(s) may include carbon. In any embodiment, the working and/or counter electrodes may include glassy carbon, carbon nanotubes, graphene, gold, platinum, silver, chromium, graphite, carbon black, or a combination of two or more thereof. In any embodiment, the reference electrode material may include be silver (e.g., silver chloride), calomel electrode, standard hydrogen electrode, normal hydrogen electrode, palladium hydrogen electrode, or a combination of two or more thereof. In any embodiment, the working electrode may have a diameter of about 0.1 mm to about 5 mm.

In any embodiment, the surface of the working electrode, the counter electrode, and/or the reference electrode may be modified. In any embodiment, the surface of the working electrode may be modified. Modification includes the addition of a conductor(s) and/or a semiconductor(s) to the electrode surface. In any embodiment, the conductor(s) and/or semiconductor(s) may include carbon materials, conductive polymers, nanoparticles, or a combination of two or more thereof. Carbon materials may include carbon nanotubes (e.g., single walled and/or multiwalled), fullurenes, graphene, reduced graphene oxide, or combinations of two or more thereof. Conductive polymers may include polyaniline, polypyrrole, polythiophene, poly(3,4-ethylenedioxythiophene), poly(o-toluidine), polyacetylene, polyphenylenes, polypyrenes, polyazulenes, polynaphthalenes, polycarbazole, polyindoles, polyazepines, poly(p-phenylene sulfides), polyfluorenes, or combinations of two or more thereof. Nanoparticles may include spherical nanoparticles, nanowires, nanorods, nanourchins, nanoshells, nanocubes, nanoplates, nanoribbions, or combinations of two or more thereof. Nanoparticles may include metal(s) such as gold, silver, platinum, chromium, palladium, or combinations of two or more thereof. Non-limiting modes of adding the conductor(s) and/or the semiconductor(s) to the electrode surface include depositing the modifying material by way of physical deposition (e.g., drop cast, spin cast, or screen printed) and/or electrochemical deposition (e.g., electropolymerization of a polymer or reduction of a carbon material). In any embodiment, the surface modification may improve the mechanical, chemical, and/or electronic interface.

In any embodiment, the device may include a body that includes a capsule that encapsulates a solvent and chamber configured to receive the consumable good sample. In any embodiment, the device may further include a substrate with a consumable good sample on the surface configured for insertion into the chamber. In any embodiment, the chamber may also provide an area for mixing the solvent with the consumable good sample. In any embodiment, the body may at least partially surround the sensor, capsule, and chamber. In any embodiment, the body may at least partially surround the sensor, capsule, chamber, and substrate. In any of the above embodiments, the device further comprises a recess configured to house the sensor. In any embodiment, the body may be a multi-use body. In any embodiment, the body may be a one-time use body. In any embodiment, the body may be disposable. In any embodiment, the body may be recyclable. A typical disposable body may contain multiple sensors, including one or more first electropolymerized chips and/or one or more second electropolymerized chips. In any embodiment, the sensor may include one or more additional electropolymerized chips that include an MIP of another trace molecule different from the trace molecule of the first electropolymerized chip.

In any of the above embodiments, the device may further include a locking mechanism for locking the substrate into the chamber. In any of the above embodiments, the locking mechanism may include a ramp adjacent to the chamber. In any of the above embodiments, the ramp is configured to puncture the capsule releasing the solvent into the chamber.

In any embodiment, after exposing the sample to the substrate, the substrate may exposed to the sensor. Exposure may be direct or the substrate may first be exposed to a liquid solvent that in turn solubilizes, extracts, mixes, and/or encourage selective binding of the potential tracer molecule from the sample. Alternatively, the solvent may be used to reduce the solubility of a tracer molecule, altering the equilibrium between being dissolved in the solvent and bound to the imprinted polymer. In any embodiment, the solvent(s) may be stored in compartments, capsules, or pouches inside a disposable unit. In any embodiment, the body may include a capsule that encapsulates the liquid solvent. In any embodiment, the solvent may include water, aqueous buffer, an electrolyte solution, an organic solvent (e.g., ethanol), or a combination of two or more thereof. In any embodiment, the solvent may include an aqueous buffer. In any embodiment, the aqueous buffer may include a mild alkaline buffer solution (pH ˜9-11 carbonate/bicarbonate). In any embodiment, the solvent may include an electrolyte solution (e.g., potassium chloride solution). In any embodiment, the device may include a chamber for mixing the solvent with the consumable good sample. In any embodiment, the sample may be incubated with the solvent (e.g., from about 1 second to 30 minutes, from about 2 seconds to 10 minutes, or from about 5 seconds to 5 minutes). Incubation to allow for trace molecule binding and electrochemical probing may be separate events, or they may happen simultaneously. Under simultaneous conditions, the solvent may further include an appropriate redox probe electrolyte solution (e.g., K₄Fe(CN)₆/K₃Fe(CN)₆ and/or Ru(NH₃)₆Cl₃/Ru(NH₃)₆Cl₂). Under separate events, after incubation the sample may be moved to an appropriate redox probe electrolyte solution (e.g., K₄Fe(CN)₆/K₃Fe(CN)₆ and/or Ru(NH₃)₆Cl₃/Ru(NH₃)₆Cl₂). In any embodiment, the sample may or may not undergo purification steps such as filtration or dialysis.

In any of the above embodiments, the device may further include a locking mechanism for locking the substrate into the chamber. In any of the above embodiments, the locking mechanism may include a ramp adjacent to the chamber. In any of the above embodiments, the ramp is configured to puncture the capsule releasing the liquid into the chamber.

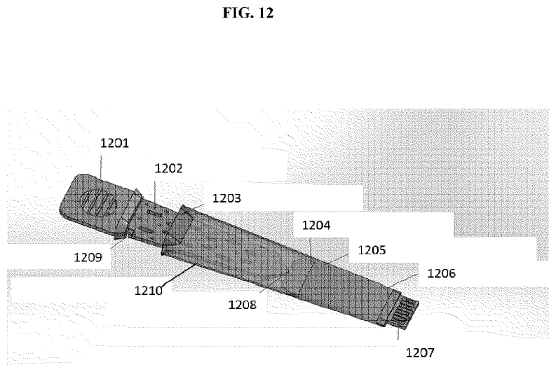

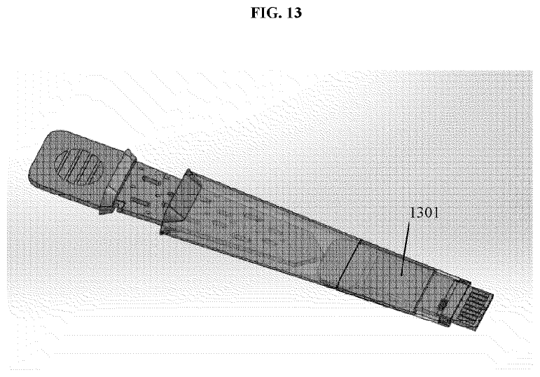

FIG. 12 shows an illustrative embodiment of a device with a locking mechanism that can lock the substrate into a chamber. Referring to FIG. 12, the substrate may be a single molded strip 1201 that includes channels for capillary action on liquids and cheese grater to capture hard/dry food 1202. The body 1210 may have an overmolded rubber 1203 that seals against strip when completely inserted into the chamber. The chamber of FIG. 12 may contain a plastic pouch 1204 filled with a liquid solvent. Upon insertion of the substrate strip into this chamber, the substrate strip punctures the plastic pouch with a pointed tip 1208 and the solvent is released and mixes with the sample. FIG. 12 also illustrates how a locking mechanism 1209 may ensure seal and prevent reuse. After mixing the sample with the solvent, the sample may be exposed to the printed circuit board inside the printed circuit board holder 1206. The printed circuit board terminates in a MicroSD connector 1207 for insertion into the Amulet processing device. FIG. 13 shows inside view of the device and the printed circuit board 1301 inside the device.

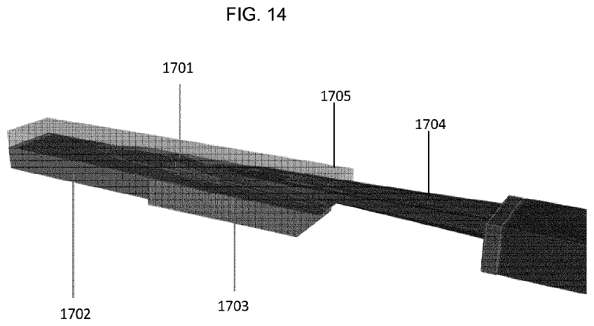

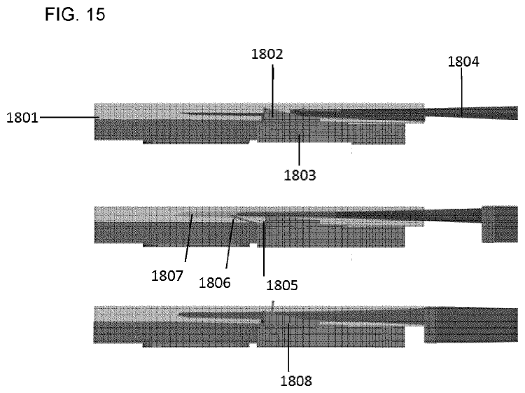

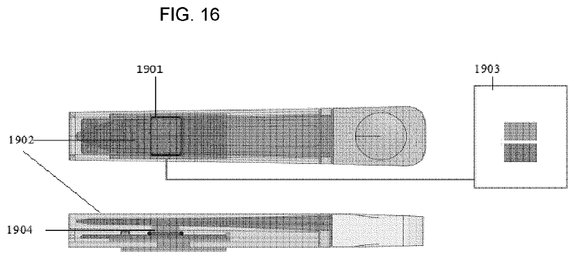

In another embodiment, the solvent may be released by a ramp. In some embodiments, the ramp is adjacent to the chamber. In some embodiments, the compression of the ramp punctures the capsule releasing the solvent into the chamber. Referring to FIG. 14, a ramp 1701 may be pushed down by the insertion of a substrate strip 1704 into the body 1705. The solvent is then released from the chamber 1703 under the ramp, and the solvent flows over the printed circuit board 1702. FIG. 15 illustrates in another embodiment how inserting the substrate strip 1804 into a body 1801 pushes down a ramp 1802 to release a solvent from a chamber 1803 by breaking a seal 1805, resulting in that a solvent 1806 flows into a chamber 1807. FIG. 15 also illustrates how the ramp 1802 may function to lock the strip in place 1808. Referring to FIG. 16, a body 1902 surrounds the printed circuit board with the MIP and NIP electropolymerized chips 1903, located within the body of the device at 1901 (overhead view) or 1904 (side view).

In any embodiment, the device may include a recess configured to house the sensor. In any embodiment, the recess may have a first portion for housing the sensor in a first position and a second portion for housing the sensor in a second position, the first and the second portions being linked such that the sensor is moveable from the first position to the second position in the recess. In any embodiment, when in the first position the sensor may be in contact with the chamber. In any embodiment, when in the second position the sensor may not be in contact with the chamber. In any embodiment, when in the second position a portion of the printed circuit board may be outside of the body. In any embodiment, the device may further include an access port communicating with the recess for manual movement of the sensor between the first and second positions.

In any embodiment, the device may further include a substrate. In any embodiment, the substrate may have been or may be exposed to the sample. In any embodiment, the substrate may be inserted into the chamber of the device. In any embodiment, the substrate may have an elongated shape such as in the shape of a strip or a pin. In any embodiment, the substrate may include glass, plastic, paper, quartz, alumina, mica, silicon, a III-IV semiconductor compound, or combinations of two or more thereof. FIGS. 12-13 show an illustrative embodiment of a substrate with elongated shape. In any embodiment, the substrate may have a top and a bottom surface. In any embodiment, the bottom may have a tapered end and/or the top may have a portion for holding the substrate. In any embodiment, the substrate may include one or more holes and/or cervices. In any embodiment, upon inserting the substrate into the chamber of the device may result in puncturing a capsule containing a solvent as described herein. In any embodiment, the substrate may be a multi-use substrate. In any embodiment, the substrate may be a one-time use substrate. In any embodiment, the substrate may be disposable. In any embodiment, the substrate may be recyclable. In any embodiment, the substrate may include a wireless transceiver.

In any embodiment, the sensor may be stored in a dry compartment within the disposable or in a compartment containing a solvent. In any embodiment, additional chemicals may also be mixed with the sample to modulate the solubility of the tracer molecule. Such chemicals include buffers, salts, and surfactants. In any embodiment, the chemicals may be stored in the same chamber as the sensor or in a separate chamber.

In any embodiment, the device may further include a processing device. Commonly, the processing device may be configured to communicatively couple to the sensor and may be configured to determine an electric current difference between the MIP film and the NIP film. In any of the above embodiments, the processing device may determine the presence of the allergen when the electric current of the MIP film is greater than the electric current of the NIP film. In any of the above embodiments, the processing device may determine the presence of the allergen when the electric current of the MIP film is lower than the electric current of the NIP film. In any embodiment, the electric current may be determined by cyclic voltammetry (CV), linear sweep voltammetry, square wave voltammetry, differential pulse voltammetry, amperometry, or a combination of two or more thereof. In any embodiment, the electric current may be determined by cyclic voltammetry (CV). In any embodiment, the processing device may determine the presence of the allergen when the resistance of the MIP film is lower than the resistance of the NIP film. In any embodiment, the processing device may determine the presence of the allergen when the resistance of the MIP film is higher than the resistance of the NIP film.

FIGS. 1-11 show illustrative embodiments of the processing device. In some embodiments, the processing device is configured to communicatively couple to the sensor. In some embodiments, the processing device comprises circuitry configured to determine presence of the food allergen. In another embodiment, to determine presence of the food allergen the processing device is configured to compare an electric current of the MIP to the electric current of the NIP. In further embodiments, the processing device is configured to determine an electric current difference between an electric current of the MIP and an electric current of the NIP; compare the electric current difference to a threshold difference. In further embodiments, the processing device is configured to determine that the food allergen is present when the electric current difference is greater than the threshold difference. In further embodiments, the processing device determines that the food allergen is present when the electric current of the MIP is greater than the electric current of the NIP. In yet further embodiments, the processing device determines that the food allergen is present when the electric current of the MIP is less than the electric current of the NIP.

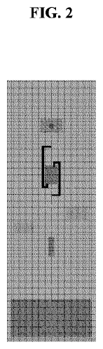

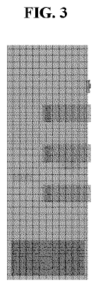

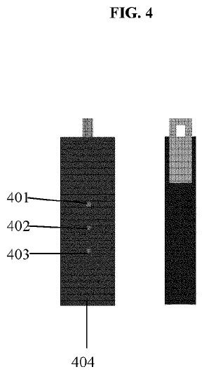

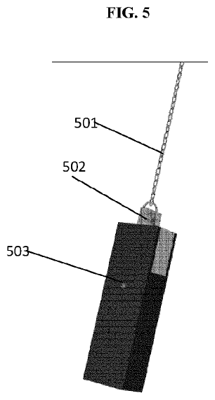

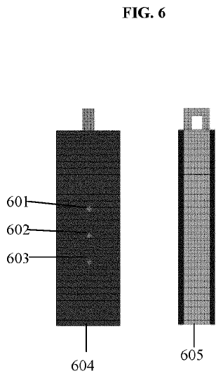

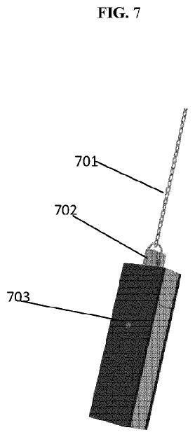

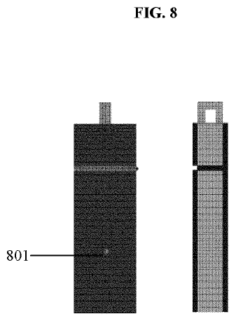

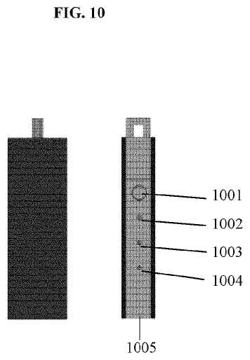

In any embodiment, the sensor may connect to the re-usable reader or a processing device (the “Amulet”). The Amulet may contain the necessary electronics (multimeter/potentiostat/microprocessor/physical memory) to analyze the chips. FIGS. 1-3 shows illustrative embodiments of the printed circuit boards of the Amulet. Referring to FIG. 1, the Amulet may have a housing include a top part 101, a short end side part 102, and a bottom part 104, that contains within a printed circuit board configured to connect to a MicroSD connector of a removable part of the food allergen detection device 105 described above. The assembled Amulet may have a push button 106 configured to initiate reading of electrical electric current in the food allergen detection device. The Amulet may further have a surface mounted green led light 107 that lights up if the food allergen is absent, a surface mounted red led light 108 that lights up if the food allergen is detected, and a surface mounted yellow light 109 that indicates reading in process by flashing and inconclusive result if the yellow light is stable. FIGS. 2 and 3 show illustrative embodiments of the circuit board for the processing device. In some embodiments, as illustrated in FIG. 4 or FIG. 6, the green light 401 or 601, the red light 402 or 602, and the yellow light 403 or 603, may be on the front side 404 or 604 of the processing device. In some embodiments, as illustrated in FIG. 10, the green light 1002, the red light 1003, and the yellow light 1003, may be on the side 1005 of the processing device. In some embodiments, the processing device may have a push button 1001 to initiate the reading of the food allergen detection device. In some embodiments, the processing device may only have one light mounted on the surface to read the result of the food allergen testing. FIG. 5 shows an embodiment of the device having one light 503 mounted on the surface. FIG. 7 shows another embodiment of a device having one light 703 mounted on the surface. FIG. 8 shows another embodiment of a device having one light 801 mounted on the surface.

The relative measurements of each chip are used to determine whether the trace molecule is present. Readings for each chip may be taken once, multiple times, or continuously. In some embodiments, the device may further comprise a processing device, wherein the processing device is configured to communicatively couple to the sensor, wherein the processing device is configured to determine an electric current difference between an electric current of the MIP film and an electric current of the NIP film. In further embodiments, the processing device determines the presence of the first food allergen when the electric current of the electric current of the MIP film is greater than the electric current of the NIP film. In another embodiment, the processing device determines the presence of the first food allergen when the electric current of the electric current of the MIP film is less than the electric current of the NIP film.

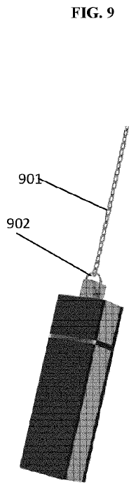

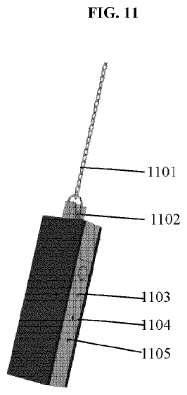

In another embodiment, the processing device communicatively couples to the sensor via a plurality of contacts of the sensor and via a plurality of contacts of the processing device. In further embodiments, the processing device is a wearable. FIGS. 5, 7, 9, and 11 show illustrative embodiments of a wearable processing device. Referring to FIG. 5, a chain 501 is connected to a hook 502 that is part of the processing device, so that the device can be placed around the neck of the subject wearing the device. Referring to FIG. 7, a chain 701 is connected to a hook 702 that is part of the processing device, so that the device can be placed around the neck of the subject wearing the device. Referring to FIG. 9, a chain 901 is connected to a hook 902 that is part of the processing device, so that the device can be placed around the neck of the subject wearing the device. Referring to FIG. 11, a chain 1101 is connected to a hook 1102 that is part of the processing device, so that the device can be placed around the neck of the subject wearing the device. In another embodiment, the processing device communicatively couples to the sensor via a wireless signal. In further embodiments, the wireless signal comprises a radio and/or infrared frequency signal. In yet further embodiments, the processing device is a computer, telephone, watch, and/or mobile device.

In another aspect, the present technology provides a method of making the allergen detection device described herein. MIPs and NIPs may be manufactured by methods known to those of skill in the art including those provided in U.S. Pat. No. 9,846,137, which is herein incorporated by reference. In any embodiment, the method may include providing a conductive electrode, depositing a polymer in the presence of the trace molecule by electropolymerization to form the electropolymerized MIP film, and depositing the polymer in the absence of the trace molecule by electropolymerization to form the electropolymerized NIP film. In any embodiment, the depositing the polymer on a first electrochemical chip in the presence of the trace molecule provides the first electropolymerized chip and/or the depositing the polymer on a second electrochemical chip in the absence of the trace molecule provides the second electropolymerized chip. The polymer may be any polymer described herein. The trace molecule is in a consumable good and may be any trace molecule described herein. In any embodiment, the first and second electropolymerized chips may take any reasonable size and pattern for measuring the electric current of the MIP and NIP films. In any embodiment, the electropolymerized chips may be used for a 2-point electric current measurement, a 4-point electric current measurement, or more complex electrochemical measurements as described herein (e.g., CV, linear sweep voltammetry, square wave voltammetry, etc.).

In general, MIP films are synthesized by combining functional monomers/polymers with a “template molecule” to provide a pre-polymerization solution, submerging an electrochemical chip in the pre-polymerization solution, and connecting the chip to a potentiostat. In any embodiment, the pre-polymerization solution may include a solvent (e.g., water, ethanol acetonitrile, acetone, tetrahydrofuran, dimethylsulfoxide, dimethylformamide, N-methylpyrollidone, N,N-dimethylacetamide, or a combination of two or more thereof). In any embodiment, the pre-polymerization solution may include a buffer (e.g., acetate buffers, carbonate buffers, citrate buffers, phosphate buffers, or a combination of two or more thereof). In any embodiment, the pre-polymerization solution may include an electrolyte (e.g., FeCl₃, KCl, tetraalkylammonium salts, LiClO₄, LiTFMS, or a combination of two or more thereof. In any embodiment, the template molecule may have a concentration ranging from nanomolar to millimolar. In any embodiment, the pre-polymerization solution may be prepared at room temperature, but may be performed at higher or lower temperatures. In any embodiment, the pre-polymerization solution is prepared at least 1 hour prior to electropolymerization to allow enough time for complexation between the monomer/polymer and the template molecule.

In any embodiment, the potential of the working electrode may be cycled through a range of voltages which causes a film to polymerize onto the electrode surface. For example, potentiostat cycles may range from about −2 V to about 2 V (including 0 to about 1 V), about 1-100 times (including about 10-30 times), at various rates such as about 1 mV/s to about 100 mV/s (including about 40 mV/s to about 60 mV/s). In any embodiment, a single chip may be polymerized at a time, or multiple chips may be connected in parallel and coated as a batch.

After a series of cycles, the template molecule may be removed from the polymer. In any embodiment, removal of the template molecule from the MIP film may be achieved by using a solvent, surfactant, buffer, electrochemistry, or a combination thereof. For example, the template molecule may be removed by rinsing it away or overoxidizing, which leaves behind an MIP film with empty molecular cavities. In any embodiment, the solvent may be any solvent capable of dissolving the template molecule but not the polymer film (e.g., methanol, ethanol, acetonitrile, THF, DMF, DMSO, etc.). In any embodiment, an appropriate surfactant (anionic, cationic, or neutral) may be added. Anionic surfactants include, but are not limited to, alkylbenzene sulfonates, fatty acid soaps, dialkyl sulfosuccinate, alkyl ether sulfates, sulfated alkanolamides, alkyl sulfates, alpha olefin sulfonates, lignosulfonates, organophosphorous surfactants, and/or sarcosides. Nonionic surfactants include, but are not limited to, ethoxylated linear alcohols, ethoxylated alkyl phenols, ethoxylatedthiols, acid ethoxylated fatty acids (polyethoxy-esters), glycerol esters, esters of hexitols and cyclic anhydrohexitols, ethoxylated amines, imidazoles, and/or tertiary amine oxides. Cationic surfactants include, but are not limited to, fatty amines, their salts and quaternary derivatives, linear diamines, amide, ester and ether amines, oxy and ethoxy amines, and/or alkanol amides. Buffers include, but are not limited to, phosphate, carbonate, acetate, and/or citrate buffers. In any embodiment, the potential at the working electrode may be used to help remove the template molecule from the MIP film. For example, cycling between −1V to 1V to extract the template molecule from the polymer film. In any embodiment, if the template molecule is a protein, the protein may be denatured and rinsed away from the polymer. As used herein, the term “template molecule” refers to a trace molecule that can be used to create receptor sites in the polymer.

By combining template molecules with polymers, a cavity remains in the polymer after removing the template molecules. The cavities complement the template molecule in size, shape, and chemical functionality. The cavities form the receptor sites for the indicator molecules of food allergens. Thus, the MIPs are solid or gel-phase polymers which were synthesized or deposited in the presence of a template molecule. NIPs are synthesized with the same processes as MIPs but without the template molecules.

The selective binding capabilities of the MIPs can be measured by incubating them in a solution of the tracer molecule and measuring how much binding occurs. In any embodiment, binding may be measured by cyclic voltammetry (CV), linear sweep voltammetry, square wave voltammetry, differential pulse voltammetry, amperometry, or a combination of two or more thereof. Binding behavior of the MIPs is compared with the NIPs. Methods of detecting binding in such systems include direct measurement of the film to observe the incorporation of bound tracer molecule. For lab-based challenge or standardization testing, the remaining tracer molecule in solution may also be used to indirectly measure binding. These measurements may be taken before and after incubation, continuously, or with some degree of mid-incubation data points. In any embodiment, changes between the pre-incubation and post-incubation measurements for the MIP and NIP control films indicate the presence or absence of the target analyte.

Electropolymerized chips may include additional components. For example, as disclosed herein the surface of the working electrode, the counter electrode, and/or the reference electrode may be modified.

The present technology provides a convenient method to detect allergens in a consumable good. In any embodiment, the present disclosure provides a method for detecting an allergen using the allergen detection device described herein, comprising exposing the sensor to the consumable good.

In any embodiment, the method of detecting an allergen, further includes: a) exposing the substrate to the consumable good; and b) inserting the substrate into the chamber. In any embodiment, upon inserting the substrate into the chamber, the substrate may puncture a capsule filled with solvent. Hence, in any embodiment the method comprising the steps of, the inserting the substrate into the chamber may puncture the capsule and release the liquid into the chamber. In any embodiment, the method may include agitating the device. In another embodiment, the agitating may include shaking.

After the consumable good sample has been inserted into the device, and the sample has been mixed with a solvent, the user moves the sensor to a second position such that a portion of the printed circuit board is outside the body of the device. Accordingly, in some embodiments, the method further comprises moving the sensor to the second position such that a portion of the printed circuit board is outside of the body of the device. Then, the user inserts the exposed portion of the circuit board into the processing device. FIGS. 4-11 shows illustrative embodiments of processing devices. Hence, in some embodiments, the method further comprises inserting the portion of the printed circuit board outside of the body of the device into the processing device.

Finally, the user can read the result of the processing device. Accordingly, in some embodiments, the method further comprises viewing the processing device results. FIGS. 4, 6, and 10 show processing devices having red, green, and yellow lights to reveal the presence food allergen. In some embodiments, when the food allergen is present the processing device displays a red light. In other embodiments, when the food allergen is absent the processing device displays a green light.

The present technology, thus generally described, will be understood more readily by reference to the following examples, which are provided by way of illustration and are not intended to be limiting of the present invention.

### EXAMPLES

The examples herein are provided to illustrate advantages of the present technology and to further assist a person of ordinary skill in the art with preparing or using the compositions of the present technology. The examples herein are also presented in order to more fully illustrate the preferred aspects of the present technology. The examples should in no way be construed as limiting the scope of the present technology, as defined by the appended claims. The examples can include or incorporate any of the variations, aspects or aspects of the present technology described above. The variations, aspects or aspects described above may also further each include or incorporate the variations of any or all other variations, aspects or aspects of the present technology.

### Example 1: Manufacturing Electropolymerized MIP and NIP Sensor Chips

Pyrrole, a template molecule, and buffer (acetate & acetic acid pH ˜5.5) were mixed to form a pre-polymerization solution. The mixture was allowed to sit for about 1 hour. A blank electrochemical chip (carbon working electrode, carbon counter electrode, carbon reference electrode) was then place in the pre-polymerization solution. Potentiostat cycles were applied from approximately 0-1 volts 20 times (about 50 mV/s) to form an MIP film on the electrochemical chip. The template molecule was then washed away from the MIP film using a carbonate buffer. An NIP film was formed on a second blank electrochemical chip following the same conditions except the template molecule was excluded.

### Example 2: Challenge Test for the Electropolymerized MIP and NIP Sensor Chips

In a typical challenge test, a baseline cyclic voltammetry (CV) experiment from −0.5 to +1 volts at a scan rate of 10-100 mV/s is conducted of the two electropolymerized chips. The chips are then submerged in a challenge solution that includes K₄Fe(CN)₆/K₃Fe(CN)₆ and Ru(NH₃)₆Cl₃/Ru(NH₃)₆Cl₂ and a second (CV) experiment is conducted. Comparison of before and after CVs is used to determine whether the tracer molecule is present in the challenge solution. Significant differences between the two films indicates that the tracer molecule is present in the test solution.

### EQUIVALENTS

While certain embodiments have been illustrated and described, a person with ordinary skill in the art, after reading the foregoing specification, can effect changes, substitutions of equivalents and other types of alterations to the compositions of the present technology as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, or compositions, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

1. An allergen detection device comprising a sensor, wherein the sensor comprises:
a circuit board;
an electropolymerized molecularly imprinted polymer film (MIP) comprising receptor sites imprinted in a first surface of the polymer, the receptor sites configured to accept a trace molecule of an allergen; and
an electropolymerized non-imprinted polymer film;
wherein the sensor is configured to detect the presence of the trace molecule upon binding to one or more of the receptor sites on the MIP.

2. The device of claim 1 further comprising a first electrochemical chip and a second electrochemical chip, wherein the first electrochemical chip comprises the MIP and the second electrochemical chip comprises the NIP.

3. (canceled)

4. The device of claim 2 further comprising a circuit board comprising the first electrochemical chip and the second electrochemical chip.

5. The device of claim 1, wherein the device further comprises a processing device, wherein the processing device is configured to communicatively couple to the sensor, wherein the processing device is configured to determine an electric current difference between the MIP film and the NIP film.

6. The device of claim 5, wherein the processing device determines the presence of the allergen when the electric current of the MIP film is greater than the electric current of the NIP film.

7-9. (canceled)

10. The device of claim 1, wherein the polymer comprises polymerized monomers, wherein the monomers comprise 3-aminophenyl boronic acid, 4-aminophenyl boronic acid, 2-hydroxyphenyl boronic acid, 3-hydroxyphenyl boronic acid, 4-hydroxyphenol boronic acid, pyrrole, polyaniline, thiophene, 3,4-ethylenedioxythiophene, phenylene diamine, phenyl boronic acid, p-aminothiophenol, aminophenol, p-phenyl phenylenediamine, o-toluidine, or combinations of any two or more thereof.

11. The device of claim 1, wherein the trace molecule is in a consumable good.

12. The device of claim 11, wherein the consumable good comprises food, drink, cosmetic, or a combination of two or more thereof.

13. The device of claim 11, further comprising a chamber, wherein the chamber comprises a capsule that encapsulates a solvent and the chamber provides a space for mixing the solvent with the consumable good.

14-20. (canceled)

21. The device of claim 1, wherein the trace molecule comprises an organic molecule.

22. The device of claim 21, wherein the organic molecule has a molecular weight of less than about 900 Daltons.

23. The device of claim 22, wherein the organic molecule is selected from lactose, galactose, amygdalin, juglone, biochanin A, resveratrol daidzein, daidzin, genistein, and genistin.

24. The device of claim 22, wherein the organic molecule is not cortisol, an amino acid, theophylline, and/or chlorpyrifos.

25. The device of claim 21, wherein the organic molecule comprises a polypeptide, protein, epitope, aptamer, or a combination of two or more thereof.

26. The device of claim 25, wherein the organic molecule comprises at least one protein.

27. The device of claim 26, wherein the organic molecule comprises at least two different proteins.

28. The device of claim 25, wherein the organic molecule comprises at least one epitope.

29. The device of claim 28, wherein the organic molecule comprises at least two different epitopes.

30. The device of claim 25, wherein the organic molecule comprises at least one protein and at least one epitope.

31. A method of making the allergen detection device of claim 1, the method comprising:
providing a conductive electrode;
depositing a polymer in the presence of the trace molecule by electropolymerization to form the electropolymerized MIP film; and
depositing the polymer in the absence of the trace molecule by electropolymerization to form the electropolymerized NIP film.

32-46. (canceled)
